(19) United States
(12) Patent Application Publication
(10) Pub. No.: US 2007/0166294 A1
(43) Pub. Date: Jul. 19, 2007

(54) Method for reducing aflatoxins in fodders, meats, milk and derivatives thereof and a composition suitable for said purpose

(76) Inventors: Mogna Giovanni, Novara (IT); Strozzi Gian Paolo, Novara (IT)

Correspondence Address: PEARNE & GORDON LLP, 1801 EAST 9TH STREET, SUITE 1200, CLEVELAND, OH, 44114-3108, US

(21) Appl. No.: 10/590,255

(22) PCT Filed: Feb. 25, 2005
(86) PCT No.: PCT/IB05/00484

§ 371 Date: Aug. 22, 2006

(30) Foreign Application Priority Data
Feb. 27, 2004 (IT) MI2004A000357

(51) Int. Cl. A61K 35/74; C12P 1/00; A23K 1/00

(52) U.S. Cl. 424/93.45; 426/53; 435/41

(57) ABSTRACT

The invention relates to a method for reducing drastically and/or eliminating aflatoxins in feeds, thus enabling to produce meat and milk free from aflatoxins, by using specific lactic bacteria eliminating the presence of molds from feeds for cattle nutrition and the subsequent aflatoxin contamination of meats, milk and dairy products; the invention also provides a composition suitable to said purpose.

The present invention relates to a method for reducing drastically and/or eliminating aflatoxins in feeds, thus enabling to produce aflatoxin-free meat and milk. In particular, according to the method of the invention specific lactic bacteria should be used for eliminating or at least reducing to a high extent the presence of molds in cattle feeds and the related aflatoxin contamination of meat, milk and dairy products. The invention also relates to a composition for implementing the method according to the invention.

### FRAMEWORK OF THE INVENTION

Mycotoxins are contaminating substances produced by molds and quite widespread in foods and zootechnical products; mold contamination can be of primary type if occurring in fields during cultivation, or of secondary type if occurring in one of the following stages (harvest, drying, preservation, transformation, transport, etc.).

This phenomenon is so widespread all over the world that in 1985 FAO estimated that about 25% of foodstuffs in the world was contaminated.

The main factors determining and affecting the production of mycotoxins are:

Intrinsic factors:
the initial level of contamination in food fungi spores, which affects the amount of synthesizable toxins (the more molds the higher potential amount of mycotoxins);
the contaminating fungi species, which determines the classes of mycotoxins produced;
the toxigenic potential, which is very different for stocks belonging to the same species.

Extrinsic factors:
general environmental and ecological conditions;
specific chemical, physical-chemical and physical food-related factors, such as humidity, free water (a_{w}), temperature, nature of substrate, partial oxygen pressure, which affect fungi development and therefore mycotoxin production;
biological factors, which are essentially referable to the competition exerted onto molds by the natural microflora that is present in food or that can be added so as to control fermentation for food preservation.

Out of about 800 classified mycotoxins, aflatoxins are the most toxic for animals and humans; said group now comprises 18 different substances with different toxicity affecting liver, kidney, nervous system as a consequence of ingestion, skin contact and inhalation. These substances are essentially produced by Aspergillus flavus (ubiquitous), Aspergillus parasiticus (more frequent in subtropical and tropical climates) and Aspergillus nonius (less frequent). The most frequent aflatoxins in foodstuffs are B1, B2, G1 and G2.

The most dangerous among these is aflatoxin B1, classified by the IARC (International Agency for Research on Cancer) as carcinogenic since, beyond having a direct effect, it is metabolized in the animal's organism and transformed into aflatoxin M1, which gets into milk passively through the cell membrane.

Foods undergoing aflatoxin B1 contamination most frequently are corn, oil seeds, cereals and legumes. Aflatoxin M1, though less toxic than its precursor B1, is also classified as potentially carcinogenic.

When cows ingest feeds contaminated by aflatoxin B1, the latter is transformed through hydroxylation in the liver and kidneys into aflatoxin M1, a small part of which is deposited into the animal's meats, whereas almost the whole of it is then eliminated into milk. Its appearance in milk begins about 12 hours after contaminated feeds have been administered and reaches its maximum concentration in about 24 hours; it disappears about 24-72 hours after contaminated feeds are removed from the diet.

There is quite a precise correlation between concentration of aflatoxin B1 in cattle feeds and concentration of aflatoxin M1 in meats and milk.

In particular, it has been calculated that the transfer ratio of aflatoxin B1 into feed to M1 into milk is of about 55:1 (Frobisch, 1986).

Considering that aflatoxin M1 is not deactivated by heat treatments, it can be inferred that contamination extends also to all dairy products. Indeed, cheese represents a still higher danger for consumers' health, since it has a higher concentration of toxin M1 than processed milk; this phenomenon is due to the particular chemical affinity of aflatoxin towards proteins.

Alarming episodes of contamination of milk and dairy products are more and more frequent in every country and it is therefore more and more urgent to contrast this phenomenon drastically; being maize silages the main portion of cattle nutrition, they constitute the primary source of the genesis of aflatoxin M1 in the whole milk production chain.

It is therefore absolutely necessary to interrupt contamination since its origin.

Norms concerning mycotoxins in foods for human nutrition (EC Regulation 644/2001 issued by the Commission on Mar. 8, 2001, hereinafter referred to only as “Regulation”) establish as the maximum amount of M1 in raw milk, milk for nutrition and milk for the production of dairy products a concentration of 0.05 ppb (μg/kg), whereas the limit imposed by the Regulation for feeds for dairy animals is of 5 ppb.

Actually, referring to maize silage, said limit should be far lower. Indeed, considering the limit of 0.05 ppb in milk, a consumption of about 20 kg of silage/die/animal and a daily production of 30 liters of milk, the result is:

0.05 ppb×30 (liters of produced milk/die)=1.5 μg (total aflatoxin M1 eliminated into milk)

1.5 μg×55 (transfer ratio)=82.5 μg (total aflatoxin B1 received with diet)

82.5 μg: 20 kg (average amount of consumed silage/die)=4.1 ppb (concentration of aflatoxin B1 in silage)

However, considering that the amount of produced milk can be less than 30 liters/die, that the amount of silage consumed with the diet can be more than 20 kg/die and that milk transfer ratio (B1↓M1) can be above 55, in order to meet the limits imposed by the Regulation of 0.05 ppb of M1 in milk with a certain safety, silages with a concentration of B1 above 2.5-3 ppb should not be administered to cows.

Due to the dangerous carcinogenic-genotoxic effect of aflatoxin B1 and to a smaller extent of M1, it is anyhow mandatory, leaving aside the limits imposed by the Regulation, to reduce the concentrations thereof as much as possible.

Researches made in Italy and Europe on waxy corn silages have shown that samples are divided into classes, depending on the concentration of aflatoxin B1, in quite a inhomogeneous way:

| | |
|---|---|
| ˜28% | <2 ppb |
| ˜12% | 2-5 ppb |
| ˜12% | 5-10 ppb |
| ˜26% | 10-30 ppb |
| ˜22% | >30 ppb |

In short, more than a half of the samples tested is far beyond the limit imposed by the Regulation for feeds for dairy animals (5 ppb) and the situation is obviously even worse if compared to the safety threshold referred to above (2.5-3 ppb).

Examining further the distribution of the various classes, it can be inferred that the highest percentages are the extreme ones (<2 ppb and >10 ppb), which shows that, if in the silo there are no suitable conditions contrasting fungi development, the number of molds reaches very high values, with a subsequent high biosynthesis of aflatoxin B1.

The fungi species that are most widespread in silages, including waxy corn silages, are Aspergillus flavus and Aspergillus parasiticus, whose optimal growth conditions involve temperatures of 25 to 30 centigrade degrees and a relative humidity of or above 85%. Such environmental conditions are optimal not only for mold proliferation but also for the production of aflatoxin B1, whose synthesis strongly decreases at temperatures above 35° C. and below 15° C.

The main aim of ensilage is to preserve fodder for winter, limiting the loss of nutritive substances and preventing at the same time the silage from taking on such nutritional and organoleptic properties as to make its use quite poor from a nutritive point of view.

Said aim is not easily achieved since, with a generally favorable epiphyte flora brought by fodder itself, there occurs an unavoidable contamination with earth and organic residues polluted by various microbial species, some of which can reduce or alter the nutritional principles of the silage, whereas others can be pathogenic for animals and indirectly for humans as well.

Beyond silages molds develop also in other types of feeds for animal nutrition, in particular in maize grain. Grain can be “humid” (humidity content of about 24-25%) and in this case ensilage is carried out to preservation purposes, or “dried” (humidity content below 13%) and in this case harvesting is followed by a drying step with hot air until the desired humidity value is reached.

It is therefore necessary to reduce feed contamination caused by some fungi species such as Aspergillus flavus and Aspergillus parasiticus, which are strong producers of aflatoxins, in particular of B1, and are therefore a serious danger for the health of animals eating said feeds and for humans eating foods derived from said animals, in particular milk and dairy products but also meat from said animals, which can contain aflatoxin M1 even though in lower concentrations with respect to milk.

Patent application MI2001A002202 describes a method for reducing butyric swelling and/or propionic fermentation caused by clostrides and by propionic bacteria in medium- and long-seasoning cheese by means of specific treatments carried out on fodder silages for the nutrition of dairy cows.

In particular, said patent application describes the use of suitable, advantageously “activated” stocks belonging to the genus Lactobacillus, where “activated” means that lactobacilli are perfectly active and vital since they have ended their reproductive biological cycle in suitable liquid substrates short before being spread onto cut fodder.

### SUMMARY OF THE INVENTION

The aim of the present invention is to provide a method for reducing drastically aflatoxins in feeds for animal nutrition.

Another aim of the present invention is to provide a method for eliminating aflatoxin M1 in meats and milk coming from farms where cows are fed with such feeds and, therefore, in dairy products derived from said milk.

A further aim of the present invention is to provide a composition for implementing the method according to the invention.

These and other aims are achieved with the method according to the invention, which enables to eliminate or anyhow to reduce drastically the number of fungi spores responsible for the production of mycotoxins, among which aflatoxin B1, in feeds for animals nutrition, thus reducing strongly aflatoxin M1 in milk produced by cows fed with said feeds and obviously in all dairy products derived from said milk.

As a matter of fact, it has been observed that by adding to fodders suitable stocks of lactobacilli before the preservation of feeds for animal nutrition, the number of molds strongly decreases, including the species Aspergillus flavus and parasiticus.

Thus, according to one of its aspects, the present invention relates to a method for eliminating and/or reducing the number of fungi spores responsible for the production of mycotoxins in feeds for animal nutrition, in which fodders designed for the preparation of said feeds are added with at least a stock of lactobacilli chosen in the group comprising Lactobacillus plantarum LMG P-21020, LMG P-21021, LMG P-21022 and LMG P-21023 and Lactobacillus pentosus LMG P-21019, if necessary combined with one or more stocks of forced hetero-fermentative lactobacilli.

In the present invention, the terms “molds” and “fungi spores” can be used as synonyms. Said terms refer to organisms producing mycotoxins.

In the present invention, the term “mycotoxins” refers essentially to aflatoxins.

According to another one of its aspects, the method according to the invention is more specifically designed to drastically reduce aflatoxins in feeds derived from preserved fodders, in particular aflatoxin B1.

According to a further aspect of the invention, the method is designed to eliminate aflatoxins, in particular aflatoxin M1, in milk coming from farms where cows are fed with such feeds and therefore in dairy products derived from said milk.

In the present description, the most important fungi spores responsible for the production of mycotoxins include molds, in particular the species Aspergillus flavus and parasiticus.

In the present invention, the term “fodders” refers to any vegetal derivative for animal nutrition.

A particularly advantageous fodder in the method according to the present invention is maize.

According to the present invention, the term “feeds” refers to fodders that have undergone treatments for their preservation; examples of feeds according to the invention are silages and maize grain in all available forms; maize “grain” refers for instance both to the whole panicle (cob+grains) and to single grains, either whole or skinned.

Lactobacilli used for the method according to the present invention, i.e. Lactobacillus plantarum LMG P-21020, LMG P-21021, LMG P-21022 and LMG P-21023 and Lactobacillus pentosus LMG P-21019, are described in patent application MI2001A002202 referred to above; advantageously, according to the present invention, said lactobacilli are used in “activated” form as described in said application.

Forced hetero-fermentative lactobacilli, if present, can contribute to create anaerobic conditions because of the production of carbon dioxide.

Illustrative examples of forced hetero-fermentative lactobacilli are those belonging to the species Lactobacillus fermentum, Lactobacillus brevis and Leuconostoc mesenteroides, more advantageously they are chosen among the following stocks: Lactobacillus fermentum 1789, Lactobacillus brevis LBR01 and Leuconostoc mesenteroides subsp. cremoris LcM 04, which are commercially available lactobacilli.

The method according to the present invention further enables to reduce and/or eliminate the content of aflatoxins in the meat of animals fed with treated feeds.

For implementing the method according to the present invention, one or more lactobacilli stocks as referred to above are spread onto fodders before these are transformed into feeds according to the invention.

In practice, lactobacilli stocks, advantageously diluted with little water, are spread by sprinkling onto the various layers of fodders before their preservation.

For instance, in the case of silages fodders will be treated with lactobacilli before being pressed, whereas as far as for instance maize grain is concerned, the treatment could be carried out in different manners depending on the type of grain; for humid grain when the silo is being filled with panicles and/or with grains, whereas for dried grain after harvesting panicles, before the drying step.

According to a preferred embodiment of the invention, at least two lactobacilli stocks are used, chosen among the following: Lactobacillus plantarum LMG P-21020, LMG P-21021, LMG P-21022 and LMG P-21023 and Lactobacillus pentosus LMG P-21019, advantageously three of them, still more advantageously all the stocks referred to above are used in the method according to the invention. The combination of the aforesaid stocks with forced hetero-fermentative lactobacilli can be advantageous, even though not strictly necessary to the aims of the invention.

Indeed, it has been observed through various tests that the simultaneous presence of at least 3-5 of the stocks listed above in the silage mass engenders a surprising synergic effect inducing in the feed specific pH conditions, anaerobic conditions and the secretion of antimycotic substances, which are absolutely unfavorable for the germination and development of all harmful germs, referring in particular to molds responsible for the production of aflatoxin B1.

According to another preferred embodiment, fodders are sprinkled with a suspension of at least 2 billions of lactobacilli per milliliter, advantageously of about 5 billions of lactobacilli per milliliter or even more. Thus, by way of example, for the method according to the invention the average dose to be used per quintal of fodder is of about 10 to 500 ml of the suspension referred to above, advantageously of about 100 ml.

Anyway, whatever the application mode, the dose of lactobacilli to be used according to the method of the invention should be such as to establish a clear prevalence onto the proper and improper microflora of the mass to be preserved and to start an immediate and suitable fermentation.

Suitable amounts of lactobacilli are generally of 50 to 500 billions of bacteria per quintal of fodder, for instance of about 100 billions of bacteria per quintal of fodder (1 million per gram).

Different amounts can however be used depending on the type of fodder, earth and contamination conditions.

Test results have shown that the use of the combination of the lactobacilli referred to above in the method according to the invention enables to already obtain a strong acidification in the first hours after preservation beginning, with a subsequent decrease of pH much below 3.80.

Said condition, together with a good anaerobic condition obtained through a suitable preservation technique, in particular as far as silages are concerned, and improved thanks to the production of carbon dioxide developed by stocks of hetero-fermentative lactobacilli, inhibits the development of all germs endangering silage quality, which are harmful for animals' health and, considering the whole production chain, pathogenic for humans.

Referring to molds and in particular to Aspergillus flavus and parasiticus, not only do lactobacilli according to the invention determine unfavorable conditions for their development, but they also exert a direct action of specific inhibition due to the synthesis of metabolites with antimycotic activity.

Thus, the method according to the invention enables to obtain feeds with optimal nutritional and chemical-physical properties for cattle feeding, which are also safe for the health both of animals and of consumers of milk and dairy products.

In particular, the present invention enables to obtain feeds that are almost completely free from aflatoxin B1, and considering that preserved feeds, in particular silages, represent the main portion of cow nutrition, as a consequence milk produced by animals fed with said feeds is practically free from aflatoxin M1, as the experimental part below has confirmed.

According to another one of its aspects, the invention relates to a method for producing milk free from aflatoxins, in particular of aflatoxin M1, which includes feeding dairy cows with feeds obtained with the method according to the invention.

Milk and dairy products free from aflatoxins, as are obtained with the method according to the invention, are a further object of the invention.

The use of lactobacilli in the method according to the invention is also an object of the invention.

According to another one of its aspects, the invention relates to a composition of lactobacilli comprising one or more of the following lactobacilli: Lactobacillus plantarum LMG P-21020, LMG P-21021, LMG P-21022 and LMG P-21023 and Lactobacillus pentosus LMG P-21019, in combination with one or more forced hetero-fermentative lactobacilli, for instance chosen among Lactobacillus fermentum 1789, Lactobacillus brevis LBR01 and Leuconostoc mesenteroides subsp. cremoris LcM 04, for treating fodders according to the method of the present invention.

According to a preferred embodiment, the composition is in anhydrous form to be rehydrated before use.

As an alternative, the composition can be in form of liquid culture.

The following examples disclose the invention and describe the experimental tests carried out for showing the activity of lactobacilli in the method according to the invention.

### EXPERIMENTAL PART

In the following experimental part, the composition according to the invention, made up of a reactivated culture of lactobacilli stocks, is referred to as “PRODUCT”.

Said PRODUCT will also be referred to as Aflasil.

The test carried out with the PRODUCT involves a significant area designed for maize culture for a total of above 400 maize-cultivated hectares and with about 3,000 milk-producing cows in cattle sheds.

The total amount of maize, harvested at waxy ripening, cut up and ensilaged, is of about 250,000 quintals. All tests are carried out by preparing also “untreated” silages and silages “treated” with commercially available anhydrous cultures, made up of stocks of non-reactivated aspecific lactobacilli. Both “untreated” silages and silages “treated with anhydrous aspecific cultures” are used as reference.

In short, the following silages are prepared:

cut maize treated with PRODUCT;

cut maize treated with commercially available bacterial “starters” (control);

cut or piled maize “non treated” with any bacterial culture (reference).

Each PRODUCT dose in anhydrous form is rehydrated as concentrated liquid culture (10 liters) the day before use, then diluting it with water at the end of reactivation to a final volume of 50 liters. Its dispersion in the cut mass is thus made easier and more homogeneous.

After reactivation and before dilution with water, the number of bacteria in the concentrated PRODUCT culture is always above 5 billions of lactobacilli per milliliter, with a pH of about 3.80.

The average dose used per quintal of cut maize is of about 100 ml of the culture diluted with water; therefore, at least 100 billions of bacteria per quintal of silage (1 million per gram) are added so as to establish a clear prevalence onto the proper and improper microflora of the mass to be ensilaged and to start an immediate and suitable fermentation.

Each standard PRODUCT dose, corresponding to 10 liters of concentrated reactivated liquid culture and to 50 liters of ready-for-use liquid culture, is thus used for treating on average 500 quintals of silage.

Administration is carried out by directly sprinkling the vegetal mass when loading the silo, spreading the culture onto every layer of cut maize, which then undergoes a suitable compression so as to obtain convenient anaerobic conditions.

The cut mass is covered with plastic sheets adhered to said mass and perfectly sealed along the edges, so as to ensure a perfect sealing.

The same expedients are obviously applied also when preparing “untreated” silages and silages “treated” with cultures differing from PRODUCT, used according to the instructions of the manufacturing company.

Evaluation Methods for Silages Under Test

All silages, both treated and untreated, are evaluated when removed from the silo (max. 10-20 days from opening) by means of the following analyses:

Analytical Evaluations Carried Out in Loco

appearance and tastiness by visual and sensory inspection;

detection of silage smell, color and texture;

observation of anomalous factors, if present (swelling, different stratification, macroscopic presence of molds, etc.).
detection of inner temperature (30 cm depth from cut) and of product removed from silo;
measuring of pH inside the mass.
   Analytical Evaluations Carried Out in Lab on Samples of Cut Maize Taken at a Depth of 20 cm from Cutting Surface
analysis of dry substance (% d.s.);
assessment of Fodder Units (U.F.), expressed per quintal of cut maize as such;
assessment of energy concentration, expressed as U.F. per kg of d.s.;
assessment of percentage of ammonia nitrogen on total nitrogen;
evaluation of fermentative profile (AGV);
calculation of FLIEG score;
microbiological detections: yeasts, molds, clostrides;
evaluation of concentration of aflatoxins B1, B2, G1 and G2
   Results

82 samples are taken from the three types of silages prepared; in 4 cases neither control silage nor reference silage can be prepared.

Test results, expressed as an average value on single analytical evaluations, are shown in the following Table 1 and refer to cut masses treated with PRODUCT, control and reference.

TABLE 1

| | | | Silages | |
|---|---|---|---|---|
| | | Silages | treated | |
| | | treated | with other | Untreated |
| Analytical | | with | cultures | silages |
| assessments | | PRODUCT | (Control) | (Reference) |
| Appearance | | excellent | good | variable |
| Tastiness | | excellent | fair | variable |
| Inner temperature (° C.) | | 18.9 | 25.5 | 30.3 |
| Dry substance in % | | 32.7 | 31.4 | 29.6 |
| pH at removal from silo | | 3.69 | 4.18 | 4.27 |
| Fodder Units | | 27.4 | 24.6 | 22.5 |
| per quintal | | | | |
| Energy concentration | | 0.84 | 0.78 | 0.76 |
| (U.F./kg d.s.) | | | | |
| Percentage of ammonia | | 4.07 | 4.83 | 7.17 |
| nitrogen on total nitrogen | | | | |
| Fat acids in | lactic acid | 4.46 | 3.58 | 3.06 |
| % of d.s. | acetic acid | 1.83 | 1.73 | 3.41 |
| | propionic | 0.11 | 0.19 | 0.26 |
| | acid | | | |
| | butyric acid | traces | 0.11 | 0.27 |
| FLIEG score | | 92 | 76 | 32 |
| Evaluation on the basis | | 20% good | 30% poor | 20% very |
| of FLIEG score | | 10% very | 40% good | poor |
| | | good | 20% very | 40% poor |
| | | 70% | good | 30% good |
| | | excellent | 10% | 10% very |
| | | | excellent | good |
| Microbiological | clostrides | 45 | 950 | 1,100 |
| analysis | yeasts | 10³ | 10⁵ | 10⁶ |

| | |
|---|---|
| molds | See class distribution in diagram 1 |

| | |
|---|---|
| Assessment of aflatoxins | See class distribution in diagram 2 |
| B1 + B2 + G1 + G2 | |

Diagram 1: Merit class distribution of silages depending on mold number

Treatment with PRODUCT:

| | | |
|---|---|---|
| no. 28 (93%) | | <10¹ UFC/g |
| no. 2 (7%) | >10¹ | <10³ UFC/g |
| no. 0 | >10³ | <10⁵ UFC/g |
| no. 0 | >10⁵ | <10⁷ UFC/g |

Treatment “Control”:

| | | |
|---|---|---|
| no. 2 (8) | | <10¹ UFC/g |
| no. 8 (31%) | >10¹ | <10³ UFC/g |
| no. 16 (61%) | >10³ | <10⁵ UFC/g |
| no. 0 | >10⁵ | <10⁷ UFC/g |

No treatment “Reference”:

| | | |
|---|---|---|
| no. 1 (4%) | | <10¹ UFC/g |
| no. 8 (31%) | >10¹ | <10³ UFC/g |
| no. 3 (11%) | >10³ | <10⁵ UFC/g |
| no. 14 (54%) | >10⁵ | <10⁷ UFC/g |

Diagram 2: Merit class distribution of silages depending on the concentration of aflatoxins (B1+B2+G1+G2) assessed by immuno-chemical method with polyclonal antibodies (kit ELISA Ridascreen® aflatoxin total-r-Biopharm) with a maximum level of detectability of ˜1.75 ppb

Treatment with PRODUCT:

| | | |
|---|---|---|
| no. 28 (93%) | | <1.75 ppb |
| no. 2 (7%) | >1.75 ppb | <5.00 ppb |
| no. 0 | >5.00 ppb | <20.00 ppb |
| no. 0 | | >20.00 ppb |

Treatment “Control”:

| | | |
|---|---|---|
| no. 4 (15%) | | <1.75 ppb |
| no. 11 (42%) | >1.75 ppb | <5.00 ppb |
| no. 10 (39%) | >5.00 ppb | <20.00 ppb |
| no. 1 (4%) | | >20.00 ppb |

No treatment “Reference”:

| | | |
|---|---|---|
| no. 1 (4%) | | <1.75 ppb |
| no. 12 (46%) | >1.75 ppb | <5.00 ppb |
| no. 5 (19%) | >5.00 ppb | <20.00 ppb |
| no. 8 (31%) | | >20.00 ppb |

Elimination of Aflatoxin M1 into Milk

Although it is well known that aflatoxin M1 derives from a transformation of aflatoxin M1 in the animal's organism and that, therefore, by administering B1-free feeds M1-free milk is obtained, said phenomenon has however been checked with tests in loco.

For silages treated with PRODUCT, untreated silages (Reference) and silages treated with products other than PRODUCT (Control), 3 groups of cows fed with said three types of silage are identified.

After a week during which cows are fed in different ways, samples of mass milk coming from said three groups are analyzed for assessing the presence of M1 by immuno-chemical method with polyclonal antibodies (kit ELISA Ridascreen® aflatoxin total-r-Biopharm) with a maximum level of detectability of 0.005 ppb (5 ppt), i.e. 10 times more sensitive than the maximum limit allowed in Italy.

The distribution of milk merit classes depending on M1 concentration is shown in the following diagram 3:

Cows fed with silage treated with PRODUCT:

| | | |
|---|---|---|
| no. 23 (7.7%) | | <0.010 ppb |
| no. 6 (20%) | >0.010 ppb | <0.025 ppb |
| no. 1 (3%) | >0.025 ppb | <0.050 ppb |
| | | (limit imposed by |
| | | the Regulation) |
| no. 0 | >0.050 ppb | <0.080 ppb |
| no. 0 | | >0.080 ppb |

Cows fed with silage treated with other products “Control”:

| | | |
|---|---|---|
| no. 4 (15%) | | <0.010 ppb |
| no. 6 (23%) | >0.010 ppb | <0.025 ppb |
| no. 4 (15%) | >0.025 ppb | <0.050 ppb |
| | | (limit imposed by |
| | | the Regulation) |
| no. 9 (35%) | >0.050 ppb | <0.080 ppb |
| no. 3 (12%) | | >0.080 ppb |

Cows fed with untreated silage “Reference”:

| | | |
|---|---|---|
| no. 0 | | <0.010 ppb |
| no. 1 (4%) | >0.010 ppb | <0.025 ppb |
| no. 8 (31%) | >0.025 ppb | <0.050 ppb |
| | | (limit imposed by |
| | | the Regulation) |
| no. 7 (27%) | >0.050 ppb | <0.080 ppb |
| no. 10 (38%) | | >0.080 ppb |

As is well known in the field, the term forage units (U.F.) refers to the nutritional power supplied by one kilo of barley or by 2.5 kilos of normal grass hay, rich in Phleum pratense and of other grass substances. The evaluation of the fermentative profile (AGV) is expressed as the evaluation of the percentage of fat acids with respect to dry substance (d.s.).

FLIEG score is a method for evaluating the quality of silages through several parameters, especially for evaluating their preservation: an excellent silage should have a score of 90-100.

FLIEG score is calculated:

1) by assessing the percentage of fat acids with respect to dry substance (AGV);

2) by calculating the milli-equivalents of each acid;

3) by adding up the milli-equivalents and expressing the various acids taken into consideration as percentage of the sum;

4) a score of FLIEG table corresponds to each percentage;

5) by adding up the score and expressing a quality judgment.

1. A method for eliminating and/or reducing the number of molds responsible for the production of mycotoxins in feeds, characterized in that fodders for preparing said feeds are added with at least a stock of lactobacilli chosen from the group comprising Lactobacillus plantarum LMG P-21020, LMG P-21021, LMG P-21022 and LMG P-21023 and Lactobacillus pentosus LMG P-21019, if necessary in combination with one or more forced hetero-fermentative lactobacilli.

2. The method according to claim 1, characterized in that said forced hetero-fermentative lactobacilli are chosen among those belonging to the species Lactobacillus fermentum, Lactobacillus brevis and Leuconostoc mesenteroides.

3. The method according to claim 2, characterized in that said forced hetero-fermentative lactobacilli are chosen among the following stocks: Lactobacillus fernentum I 789, Lactobacillus brevis LBR01 and Leuconostoc mesenteroides subsp. cremoris LcM 04.

4. The method according to claim 1, characterized in that said mycotoxins are aflatoxin B1.

5. The method according to claim 1, characterized in that said molds are of the genus Aspergillus.

6. A method for producing cow milk free from aflatoxin M1, characterized in that dairy cows are fed with feeds prepared starting from fodders treated with at least a stock of lactobacilli chosen from the group comprising Lactobacillus plantarum LMG P-21020, LMG P-21021, LMG P-21022 and LMG P-21023 and Lactobacillus pentosus LMG P-21019, if necessary in combination with one or more forced hetero-fermentative lactobacilli.

7. The method according to claim 1, characterized in that at least two or more of said lactobacilli are used, if necessary in combination with one or more forced hetero-fermentative lactobacilli.

8. The method according to claim 1, characterized in that said lactobacilli are added to said fodders in an average dose of use per quintal of fodder of about 50 to about 500 billions of bacteria.

9. The method according to claim 8, characterized in that said dose is of about 100 billions of bacteria per quintal of fodder.

10. The method according to claim 1, characterized in that said lactobacilli are used in liquid culture.

11-16. (canceled)

17. Milk and dairy products free from aflatoxins, obtained with the method according to claim 6.

18. Milk and dairy products free from aflatoxin M1 obtained with the method according to claim 6.

19. A composition of lactobacilli comprising one or more lactobacilli chosen from the group comprising Lactobacillus plantarum LMG P-21020, LMG P-21021, LMG P-21022 and LMG P-21023 and Lactobacillus pentosus LMG P-21019 in combination with one or more forced hetero-fermentative lactobacilli, for treating fodders.

20. The composition according to claim 19, characterized in that said one or more forced hetero-fermentative lactobacilli are chosen among those belonging to the species Lactobacillus fermentum, Lactobacillus brevis and Leuconostoc mesenteroides.

21. The composition according to claim 20, characterized in that said one or more forced hetero-fermentative lactobacilli are chosen from the groups comprising Lactobacillus fermentum I 789, Lactobacillus brevis LBR01 and Leuconostoc mesenteroides subsp. cremoris LcM 04.

22. The composition according to claim 19 in anhydrous form.

23. The composition according to claim 19 in form of liquid culture.

24-27. (canceled)
